(19) United States
(12) Patent Application Publication
(10) Pub. No.: US 2016/0377611 A1
(43) Pub. Date: Dec. 29, 2016

(54) CHEMICAL SENSOR USING MOLECULARLY-IMPRINTED SINGLE LAYER GRAPHENE

(71) Applicant: POLESTAR TECHNOLOGIES, INC., Needham Heights, MA (US)

(72) Inventors: Yufeng Ma, Needham Heights, MA (US); Anni Siitonen, Needham Heights, MA (US)

(21) Appl. No.: 15/196,327

(22) Filed: Jun. 29, 2016

Related U.S. Application Data
(60) Provisional application No. 62/185,947, filed on Jun. 29, 2015.

(51) Int. Cl. G01N 33/543; G01N 27/04

(52) U.S. Cl. CPC G01N 33/54393; G01N 33/5438; G01N 27/04; G01N 2600/00

(57) ABSTRACT

Sensing methods are sought to overcome the disadvantages—poor sensitivity and selectivity, poor long-term stability, and undeployable. Sensor of these teachings for detecting and recognizing target molecules includes a layer of molecularly imprinted polymer disposed on a single layer graphene sheet. In some instances, the sensor of these teachings also includes a sensing circuit configured to detect and report impedance changes in a layer of molecularly imprinted polymer disposed on a single layer grapheme sheet, the impedance changes caused by the binding of the target molecules to the molecularly imprinted polymer.

### CROSS-REFERENCE TO RELATED APPLICATIONS

This application claims priority of U.S. Provisional Application Ser. No. 62/185,947, filed Jun. 29, 2015, entitled CHEMICAL SENSOR USING MOLECULARLY-IMPRINTED SINGLE LAYER GRAPHENE, which is incorporated herein by reference in its entirety for all purposes whatsoever.

### STATEMENT REGARDING FEDERALLY SPONSORED RESEARCH OR DEVELOPMENT

This invention was made partially with U.S. Government support from the U.S. Air Force under Contract No. FA8650-14-M-5078. The federal government may have certain rights in the invention.

### BACKGROUND

These teachings relate generally to chemical sensor using molecularly-imprinted sensor and single layer graphene.

Molecular recognition is fundamental to a number of biological mechanisms. Sensors for molecular recognition are referred to herein as chemical sensors, although that name should not be considered limiting. One example of molecular recognition is

A sensor typically has two components to recognizing element that interacts with the target molecule and a transducing element that converts the interaction into a quantifiable effect. Some common recognizing elements are based on antibody, enzymatic, cellular or bio receptor interactions. Typical transducing elements are electrochemical, optical and electric elements.

Although a number of chemical sensors configured as described above have been used, there are some basic disadvantages—poor selectivity and sensitivity, poor long-term stability, and undeployable. There is a need for chemical sensors that will overcome these disadvantages.

In order to provide a concrete example, nations face an existential threat from the intersection of terrorism and weapons of mass destruction. Chemical-warfare (CW) agents, Biological-Warfare (BW) agents, explosive materials, and toxic industrial chemicals/materials (TIC/TIM) are among those compounds that homeland security experts expect to be utilized in future terrorist attacks. For instance, the time-weighted average (TWA) exposure limits for CW agents are ˜10⁻⁷-10⁻⁵ ppm for nerve agents, ˜10⁻⁴-10⁻¹ ppm for blister (vesicant) agents, ˜10⁻¹-10¹ ppm for pulmonary (choking) agents, and ˜10⁻¹-10¹ ppm for blood agents. Providing a real-time and sensitive monitoring technology towards these agents is significantly important for both civilian and military personnel deployed around the world. Such detection technologies will allow shortened times for assessment of damage, comprehension of causal relationships and traceability, determination of actions for remediation, and notification of the affected populations.

Development of chemical and biological sensors has been considered as a priority by the DoD Joint Chemical and Biological Defense Program (JPEO-CBD) because of several operational advantages, such as: 1) capability of real-time monitoring; 2) applicability for miniaturization; 3) little to no power consumption; 4) appropriate simplicity of maintenance; 5) unobtrusive detection; and 6) highly deployable. The current JPEO-CBD research topic (CBD14-102) calls for advanced chemical/biological sensors based on radio frequency (RF) technology and graphene. The relevant key requirements are listed below along with the technical attributes needed to satisfy these requirements.
High detection probability (PD)>95% • Requires very high sensitivity
Low false alarm (FA)<5% • Requires high selectivity
Continuous monitoring • Requires long-term stability, self-recover ability, and low power consumption.

Recent development of chemical/biological sensors has been focused on sensitivity and response time by using nanomaterials and new transducers. For instance, a graphene-based chemical sensor was developed for detection of individual gas (NO2) molecules because of the outstanding carrier mobility and the excessive large surface area per unit mass. The use of high frequency-radio frequency (RF) also can increase the sensitivity by suppressing the transformation (e.g., polarization due to the applied high voltage) of target molecules and water masking effects under direct current (DC) or low frequency.

Although improvements in sensor sensitivity and response speed have been demonstrated using new transducer designs and sensing nanomaterials, high selectivity of chemical detection and adequate long-term stability of sensors are still the most significant challenges. The low selectivity is due to the fundamental nature of interactions between vapors and sensing materials that does not provide molecular recognition selectivity similar to that in biomolecular interactions. The functionalization of sensing materials with bio-recognition receptors (e.g., peptide, DNA or enzyme) is possible but decreases the long-term stability and lacks self-recover ability.

TABLE 1
Comparison of recent advances in
development of sensors for gas applications

| | | | Self- | Continuous |
|---|---|---|---|---|
| Sensor type | Sensitivity | Selectivity | recovery | monitoring |
| Polypyrrole film^{[5]} | 8 ppm | Poor | Yes | Yes |
| Inkjet-printed | 1 ppm | Poor | Yes | Yes |
| polyaniline | | | | |
| nanoparticles^{[6]} | | | | |
| Polypyrrole-coated | 60 ppb | Poor | Yes | Yes |
| TiO₂/ZnO | | | | |
| nanofibers^{[7]} | | | | |
| SnO₂ films^{[8]} | 500 ppm | Poor | Yes | Yes |
| In₂O₃-based ceramic | 15 ppm | Poor | Yes | Yes |
| sensors [9] | | | | |
| Carbon nanotube^{[10]} | 100 ppt | Poor | Yes | Yes |
| Amplifying fluorescent | 6 ppt | Good | Poor | No |
| polymer sensors^{[11]} | | | | |
| Graphene sheet^{[2]} | Single | Poor | Yes | Yes |

Table 1 summarizes the recent attempts in sensor development with their performances (with regards to achieving the DoD goals mentioned above). None of the methods of Table 1 have so far shown the attributes needed to satisfy all the requirements.

### BRIEF SUMMARY

Sensors that combine the selectivity of molecularly-imprinted polymer (MIP) with the sensitivity of a single-layer graphene sheet, providing a synergetic combination of superior sensitivity with high selectivity, are disclosed herein below.

In one or more embodiments, the sensor of these teachings for detecting and recognizing target molecules includes a layer of molecularly imprinted polymer disposed on a single layer graphene sheet. In some instances, the sensor of these teachings also includes a sensing circuit configured to detect impedance changes in a layer of molecularly imprinted polymer disposed on a single layer grapheme sheet, the impedance changes caused by the binding of the target molecules to the molecularly imprinted polymer.

In one of more embodiments, the method of these teachings for fabricating a sensor including molecular imprinted polymer functionalized graphene includes attaching electrodes to both ends of a graphene sheet, depositing and imprinting template molecules on a surface of the graphene sheet, depositing a layer of a monomer on the surface of graphene sheet, polymerizing the monomer to form the molecular imprinted polymer and removing the template molecules. In one instance, depositing the layer of the monomer and polymerizing the monomer occur in the same step. In one embodiment, the deposition and polymerization occur by electrochemical polymerization (see, for example, Y. Liu, L. Zhu, Z. Luo, H. Tang, Sen. Actuators B 185 (2013) 438, which is incorporated by reference herein in its entirety and for all purposes).

A number of other embodiments are also disclosed.

For a better understanding of the present teachings, together with other and further objects thereof, reference is made to the accompanying drawings and detailed description and its scope will be pointed out in the appended claims.

### BRIEF DESCRIPTION OF THE DRAWINGS

FIG. 1 shows a graphical schematic representation of the Sensor these teachings based on MIP Functionalized Graphene;

FIG. 2 is a graphical schematic representation of Experimental Setup for Testing Sensor Response with DC Voltage as used in these teachings;

FIG. 3 show a Step-by-step Process of Transferring Single Sheet Graphene onto Other Substrate as used in these teachings;

FIGS. 4a, 4b show a Test fixture for RF characterization of MIP-single layer graphene sensor of these teachings, shown in FIG. 4a;

FIG. 5a shows a Schematic of Radio Frequency Identification System;

FIGS. 5b, 5c show an exemplary backscattering tag;

FIGS. 6a, 6b show a Design of the Antenna: Micro-strip Via-hole Balun Connected to the Dipole Antenna with the MIP-Graphene Integrated in the Middle;

FIGS. 7a, 7b show a S₁₁ Plot of MIP-Single Layer Graphene Sensor before (Dotted Blue Curve) and after (Solid Red Curve) adding ˜5 ppm Target Vapor (Methyl Salicylate);

FIG. 8 shows Change in S₁₁ Power Return Loss Peak Value as a Function of Target Vapor Concentration (Methyl Salicylate);

FIG. 9 shows S₁₁ return loss with changing conductivity of the graphene sheet;

FIGS. 10a, 10b show radiation pattern in (a) Three dimension and (b) Two dimensions;

FIG. 11a shows in-situ change in S₁₁ Power Return Loss Peak Value upon addition of analyte-malathion (0.5 ppb), air and intervening molecules-acetone (300 ppm) and water (35 ppm) as a Function of time; and

FIG. 11b shows typical S₁₁ Plot of antenna with MIP-Single Layer Graphene Sensor before (Solid Black Curve) and after (Dotted Blue Curve) adding ˜0.5 ppb malathion vapor.

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by claims.

As used herein, the singular forms “a,” “an,” and “the” include the plural reference unless the context clearly dictates otherwise.

Sensors that combine the selectivity of molecularly-imprinted polymer (MIP) with the sensitivity of a single-layer graphene sheet, providing a synergetic combination of superior sensitivity with high selectivity, are disclosed herein below.

In one or more embodiments, the sensor of these teachings for detecting and recognizing target molecules includes a layer of molecularly imprinted polymer disposed on a single layer graphene sheet. In some instances, the sensor of these teachings also includes a sensing circuit configured to detect impedance changes in a layer of molecularly imprinted polymer disposed on a single layer grapheme sheet, the impedance changes caused by the binding of the target molecules to the molecularly imprinted polymer.

In one of more embodiments, the method of these teachings for fabricating a sensor including molecular imprinted polymer functionalized graphene includes attaching electrodes to both ends of a graphene sheet, depositing and imprinting template molecules on a surface of the graphene sheet, depositing a layer of a monomer on the surface of graphene sheet, polymerizing the monomer to form the molecular imprinted polymer and removing the template molecules. In one instance, depositing the layer of the monomer and polymerizing the monomer occur in the same step. In one embodiment, the deposition and polymerization occur by electrochemical polymerization (see, for example, Y. Liu, L. Zhu, Z. Luo, H. Tang, Sen. Actuators B 185 (2013) 438, which is incorporated by reference herein in its entirety and for all purposes).

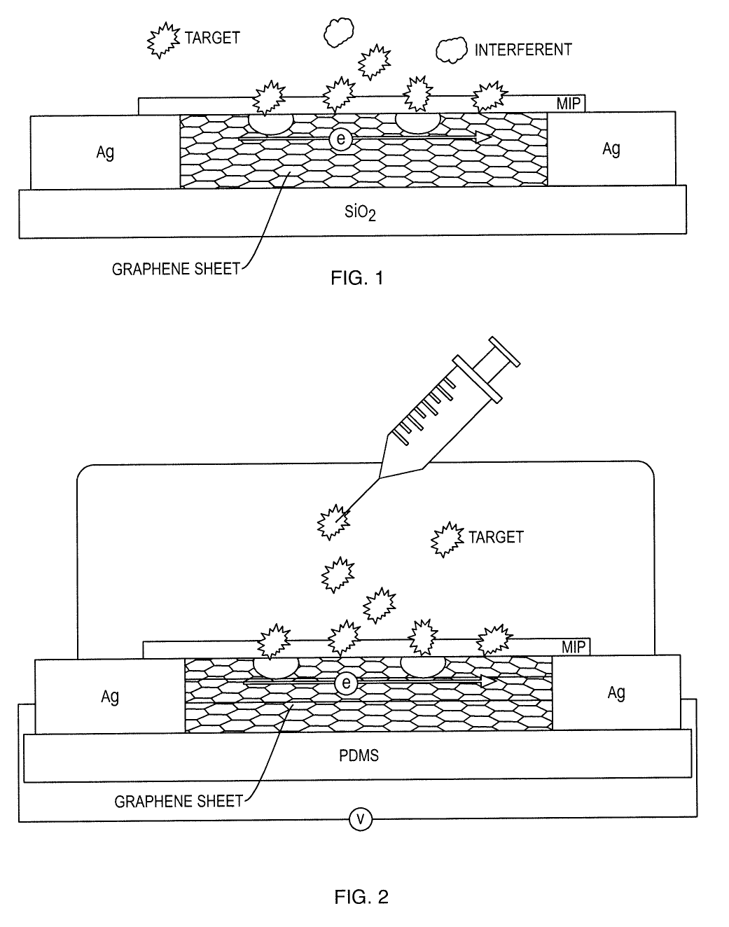

The sensors of these teachings combine selectivity of MIP with sensitivity properties of single layer graphene. The schematic of the approach is presented in FIG. 1.

High selectivity: The selectivity is introduced by the deposition of MIP. Molecular imprinting enables creation of stable and selective “artificial receptors.” Molecularly-imprinted materials have been called “antibody mimics” because these systems attempt to mimic the interactions of their natural counterparts and have achieved affinity and selectivity that approach those of natural recognitions. The technique involves the formation of complexes between a print molecule (template) and a functional monomer based on relatively weak, non-covalent interactions. These complexes appear spontaneously in the liquid phase and are then fixed sterically by polymerization with a high degree of cross-linking. After extracting the print molecules from the synthesized polymer, empty recognition sites remain in the polymer matrix. These are the “molecular recognition sites” which are able to recognize the template during subsequent rebinding procedures. MIPs are cost-effective, robust, long-term stable and are able to self-recover. Further, MIP can be applied to chemical as well as biological agents.

High sensitivity: Sensitivity is introduced by graphene. Single layer graphene has unique properties enabling detection of targets in sub parts per billion (ppb) level. Graphene is a strictly two-dimensional material and, as such, has its whole volume is exposed to surface adsorbates, which maximizes their effect. Graphene is highly conductive, exhibiting metallic conductivity and, hence, low Johnson noise even in the limit of no charge carriers², where a few extra electrons can cause notable relative changes in carrier concentration. Graphene has few crystal defects³, which ensures a low level of excess (1/f) noise caused by their thermal switching.⁴

In one embodiment, the sensing material is integrated to an antenna that allows passive and prolonged detection via Radio Frequency (RF) with low power requirement. The RF reader sends an interrogating RF signal to the sensor, an antenna, and an IC (integrated circuit) chip as a load. The IC chip responds to the reader by varying its input impedance, thus modulating the back signal. The MIP functionalized graphene sensor acts as a tunable impedance with a value determined by the existence of the target molecules. When the sensor is exposed to an analyte of interest, there is a variation in the load impedance resulting in a change in the power level. The individual sensing unit will be micro-size and numerous units can be integrated into a centimeter size chip for different targets. Such sensors can be deployed and implemented on micro-unmanned aerial vehicles, sensing network, and for stand-alone point detection.

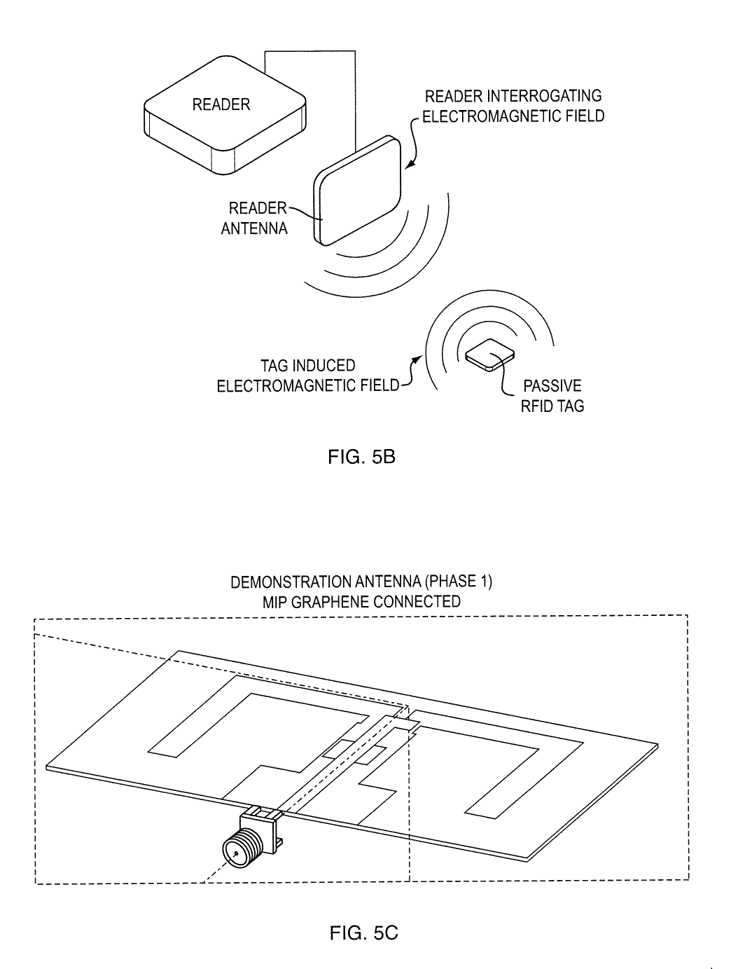

In a similar embodiment, the sensing material is used as the sensor in a backscattering tag. A sample backscattering tag is shown in FIG. 5a. Referring to FIG. 5a, the RF switch typicality a transistor. The backscatter tag includes an antenna, an RF transistor operatively connected to the antenna, a modulator/Sensing component, where the sensing component is configured to produce the data to be transmitted and the modulator is configured to receive the data to be transmitted and to toggle the RF transistor according to the data to be transmitted.(See, for example, Energy Harvesting and Sensing for Backscatter Radio, Ph. D. thesis of Spyridon-Nektarios Daskalakis, TECHNICAL UNIVERSITY OF CRETE, October 2014, incorporated by reference herein in its entirety and for all purposes).

In order to elucidate these teachings, an exemplary embodiment is presented herein below. It should be noted that these teachings that not only limited to the exemplary embodiments. In the exemplary embodiment a phenol monomer is used. In other instances, the MIP sensor is obtained by electropolymerization of o-Phenylenediamine (OPDA).

Fabrication of Sensor Unit

MIP Deposition and Optimization Taking advantage of the selectivity properties of MIP and retaining the sensitivity of graphene is a critical challenge. For this purpose, the MIP on graphene should be thin and uniform, and its fabrication condition needs to be optimized. The MIP conditions on graphene were optimized using a few sheets cleaved from Highly Ordered Pyrolytic Graphite (HOPG) with scotch tape technique. As a first step, a sample of graphene on silicone substrate with attached silver electrodes was fabricated. Silver-epoxy paste is used to attach silver wire electrodes on graphene. The optimization of the MIP condition was performed with methyl salicylate. This material is a commonly used simulant for sulphur mustard (mustard gas) due to their similar chemical and physical properties. Electrochemical deposition of methyl salicylate (pKa=9.8) on graphene was performed by applying a negative electrochemical potential to attract positively charged methyl salicylate in deionized (DI) water. The negative potential between −0.3 and −0.4 V was applied for 100 seconds while depositing 5 mM solution of methyl salicylate. The electrochemical deposition provides good control over deposition time and is faster than deposition by methyl salicylate interacting with graphene readily. The hydrolysis of methyl salicylate in water is avoided due to faster deposition time. Methyl salicylate was imprinted on graphene surface using surface imprinting technique.

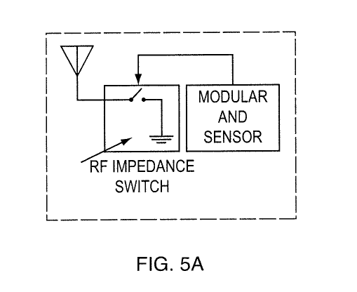

Electrochemical coating of poly-phenol (PPn) using 5 mM phenol monomer was fabricated via single cycle of cyclic voltammetry using electrochemical analyzer (CH Instruments). A three-electrode electrochemical cell was utilized to polymerize PPn. The voltage range of cyclic voltammetry was between −0.1 and 0.9 V. Silver wire was used as the reference electrode and platinum wire as the counter electrode. The fabricated MIP-graphenes were soaked in DI water overnight to remove the imprinted analytes. The parameters tested here for optimal MIP conditions were polymerization cycles, which determine the thickness of deposited PPn layer. Since the target molecules are small (molar mass of 152 g/moles) it is expected that the polymer layer should be thin to ensure the desired sensitivity. As a trade of, selectivity increases along with thickness of the MIP layer, while the sensitivity decreases. The MIP layers were optimized by varying the number of deposition cycles and the scanning rate of MIP deposition on cleaved graphene. The fabricated sensors were tested under DC voltage with a set up illustrated in FIG. 4. Current across sensor surface was monitored by CHI electrochemical analyzer before and after injecting 5 ml of methyl salicylate vapor to 25 ml container with MIP-graphene sensor, which is corresponding to ˜20 parts-per-million (ppm) concentration. The observed change in current is related to change in resistance as target molecules are attached on MIP/graphene sensor. The optimization results indicate that the best performance MIP thin layer was achieved by depositing single electrochemical cycle of PPn with scanning rate 0.03 V/second. These deposition conditions were applied in fabrication of single sheet graphene sensors.

Sensor Fabrication with Single Sheet Graphene

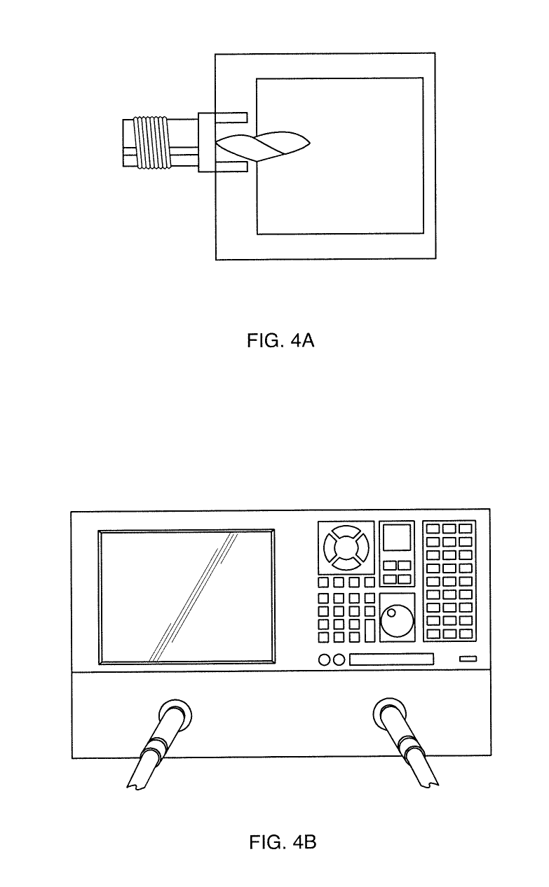

The conditions from the above embodiments on the cleaved graphene were applied onto a single layer graphene sheet. Single sheet graphene was purchased from ACS Materials (Medford, Mass.) with a size of 1 cm×1 cm. The transfer technique is provided by the manufacturer and claimed to be capable of transferring single layer graphene sheet to any substrate. The step-by-step transfer process is presented in FIG. 3. The described technique was applied for transferring the single sheet graphene onto non-conductive silicon substrate. The sample fabrication and MIP deposition was performed using conditions similar to those described in the optimization (of MIP) process.

Graphene-MIP sensor on silicon substrate was then integrated with Rogers3010 substrate with copper ground and equipped with soldered coaxial ports. A picture of the fabricated sample is presented in FIG. 4a. The fabricated samples were analyzed with a network analyzer that can be used to measure impedance changes at RF range, as shown in FIG. 4b. The network analyzer used for characterization is a broadband HP network analyzer 8510 with frequency range from 0.45 to 110 GHz. Before measurements, a full 2-port short-open-load-thru (SOLT) calibration method was carried out manually with the use of 85056K calibration standards. The network analyzer was used for characterization of graphene-MIP sensor against the resonant frequency of Power Return Loss at port 1 (S₁₁).

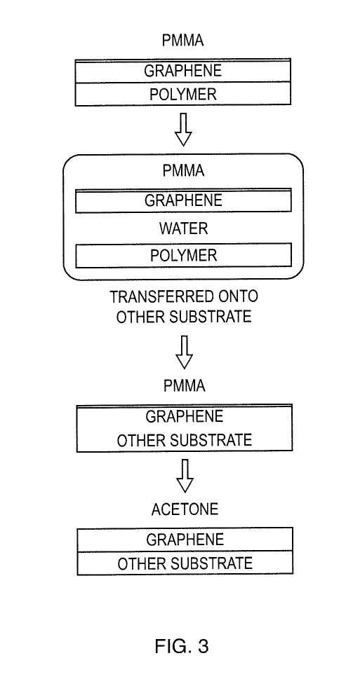

The sensitive and selective detection of target vapor, methyl salicylate, was demonstrated by following the S₁₁ peak at resonant frequency in similar test setup as described in FIG. 2 but connected to network analyzer. Different volumes of target vapor were introduced to gas chamber by injection. Measurements before and after injection were collected. After measurement, the gas chamber was purged with air for several minutes to ensure removal of target vapor as well as recovery of the sensor. The recovery was followed by allowing the S₁₁ peak to return to its original amplitude and frequency. Similar test was also performed against interfering molecules. Two molecules identified as possible interference were water vapor and acetone. Water vapor is related to changes in air humidity whereas acetone is a small organic molecule present in atmosphere as a pollutant.

Antenna Design

FIG. 5 presents a schematic of a Radio Frequency Identification (RFID) system. RFID systems use electromagnetic waves to transfer identification data at radio frequencies. A typical UHF RFID system consists of a reader system and microchip-controlled tag attached to the object to be identified. The identification data of an object is stored in the chip. If the tag is located inside the reader's interrogation zone it gets enough energy from the reader's electromagnetic fields to activate the chip, which then modulates the identification data to the carrier signal and backscatters it to the reader.

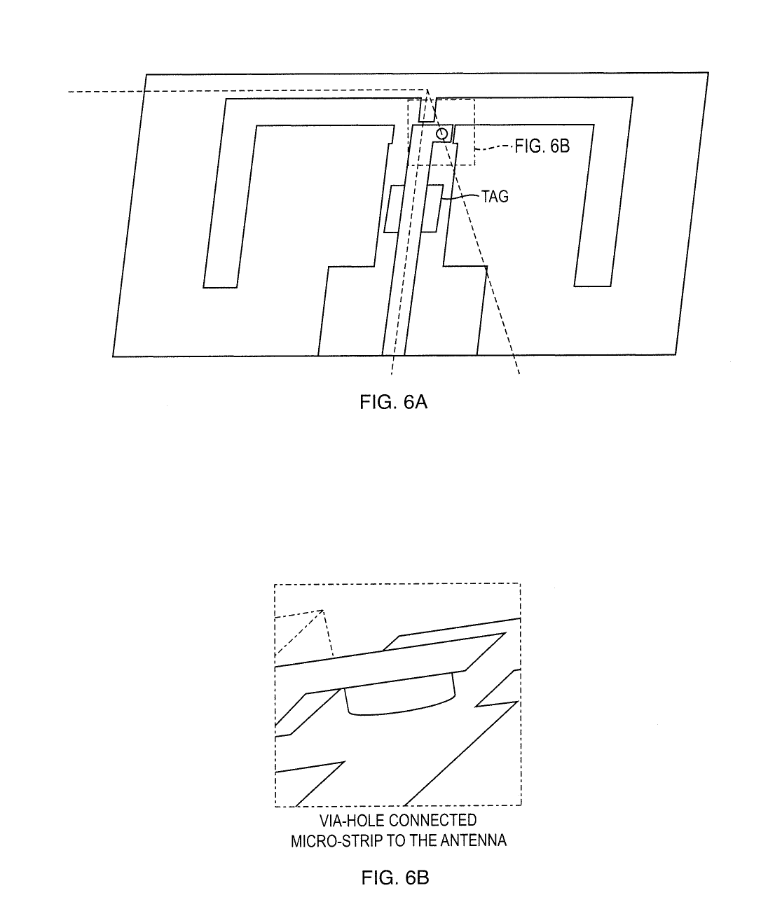

The illustration of antenna design is presented in FIG. 6. The design includes a micro-strip via-hole balun connected to the dipole antenna. The proposed design provides a stable and good matching connection between the antenna and the SMA connector. This will give us more accurate measurements with low concentration detection when using the network analyzer. Here are some key points for an RFID Tag design:
Ultra High Frequency (UHF) regulations for passive RFID in United States requires a specific frequency range: 902 MHz-928 MHz (range provided by Federal Communications Commission)
Omnidirectional radiation pattern: The omnidirectional antenna radiates or receives equally well in all directions. It is also called the “non-directional” and makes the detection range of RFID tag more flexible.
Low profile and compact size: In applications, small tag size is desirable for low fabrication costs and suitability for tag placement because free space on product packages may be very limited.
Work efficiently when mounted on a metallic object: Many applications require tag antennas to be mounted on electrically metallic objects such as vehicles and notebooks. However, while mounting on a metal, there will be degradation of the reading range and gain caused by the induced surface current on the metal.
Good Specific Absorption Rate (SAR): The biological effects of RF radiation have attracted wide interest in the research community due to the widespread of use of mobile communication systems in daily life. In order to limit the biological effects resulting from exposure to RF radiation, safety limits are usually defined in terms of the Specific Absorption Rate (SAR).

Simulations related to antenna design were performed with ANSYS HFSS 2014 (High Frequency Structural Simulator. The simulation results provide S₁₁, radiation pattern, gain analysis.

Antenna Fabrication and Integration

The antenna is fabricated on Rogers4350 substrate. It offers excellent stability of dielectric constant(εᵣ=3.66), additionally, it exhibits a low dissipation factor of 0.003 at high frequency. The exceptional electrical and mechanical stability are designed for microwave and RF applications. The wavelength λ, is defined by Equations (1) and (2).

$$\begin{matrix}λ = {c}_{0} / f \sqrt{{ɛ}_{reff}} = 21.3 cm & ( 1 ) \\ {ɛ}_{reff} = \frac{{ɛ}_{r} + 1}{2} + \frac{{ɛ}_{r} - 1}{2 \sqrt{1 + 10 H / w}} & ( 2 )\end{matrix}$$

Constant ε₀ is the speed of light in free space, ε_{reff} is the effective dielectric constant and frequency f is assumed to be 911 MHz. H is the thickness of the substrate (0.812 mm), and w is the thickness of copper antenna (0.04 mm) which is much larger than the skin depth (2.14 μm) at 911 MHz. The dimension of RFID tag is around 6 cm×10 cm.

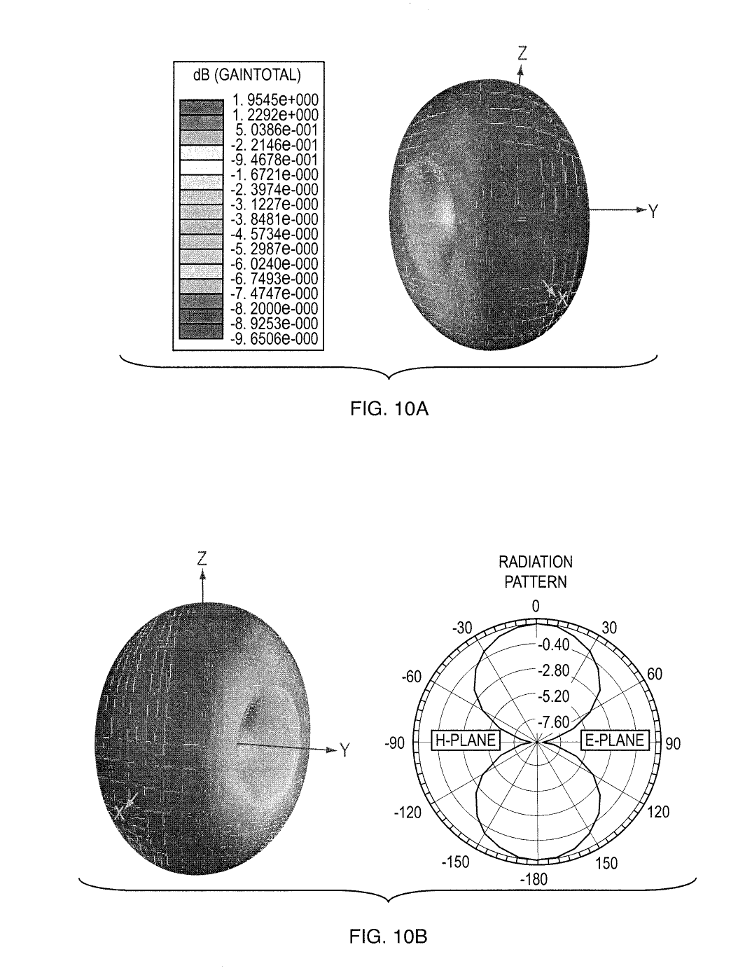

The graphene-MIP sensor will be integrated on the antenna, as illustrated in the schematic. The integration will happen in two steps. First step is to transfer single sheet graphene on antenna. A good contact between graphene and antenna is important for minimal contact resistance. After transfer the sample can be annealed for improved conductivity. The second step is to perform the MIP deposition with same parameters as before. The graphene-MIP sensor on antenna is characterized with network analyzer. FIGS. 10a, 10b show the radiation Pattern in (a) Three dimension and (b) Two dimensions. The radiation is omnidirectional which can provide the antenna radiates in 360 Degree Direction.

Results

Demonstration of Sensitive and Selective Detection of Target Vapor

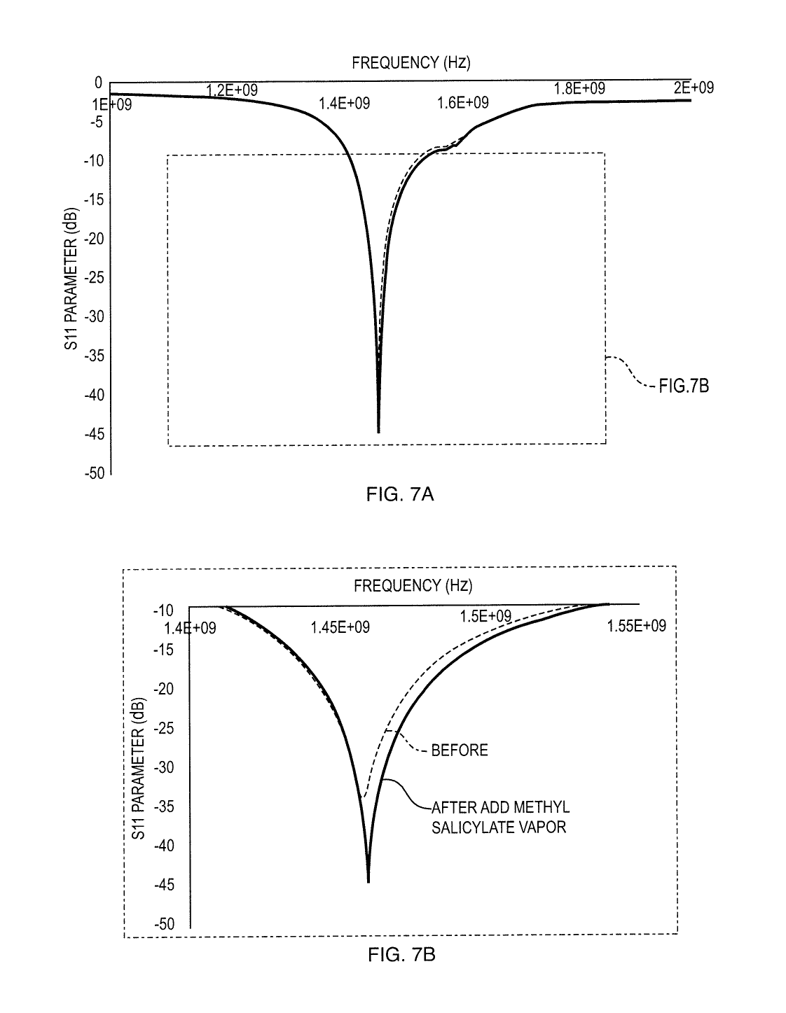

Single layer graphene was functionalized with MIP using the conditions obtained from the cleaved graphene and characterized in terms of S₁₁ parameter versus frequency. A resonant peak for the sample was identified at frequency ˜1.5 GHz. The amplitude in decibel (dB) units was followed before and after addition of target vapor. FIG. 7 shows the comparison of S₁₁ peak of graphene-MIP sensor sample before and after adding 5 ppm of methyl salicylate vapor. Upon exposure to the target vapor, the resonant frequency shifted from 1.457 GHz to 1.460 GHz while power reflection decreased from −34.5 dB to −45.1 dB. The amplitude change (dB) can be directly related to impedance change of sensor during detection of target vapor and therefore the amplitude change is of high interest.

To demonstrate the sensitive detection capability of graphene sensor, the measurements with network analyzer were performed at target vapor concentrations of 0.3-11.5 ppb. Five measurements were collected during approximately 100 second time, first measurement starting after 15 second. This time was allowing the target vapor to diffuse inside the gas chamber. After each measurement, the sample is allowed to recover in airflow for few minutes until the S₁₁ peak has returned to its original blank amplitude. The measurements were averaged and standard deviation (std) were calculated. The results are summarized in Table 2. The blank measurement with no target vapor resulted in peak amplitude of −41.4 dB with standard deviation (noise) 0.7 dB, as calculated from 5 repeated measurements. The lowest concentration tested 0.3 ppb resulted in 1.5 dB absolute change in amplitude change. As the change is more than two times the observed noise level at blank measurement. The concentration of 0.6 ppb resulted in 4.1 dB change. The results demonstrate successful gas phase detection of sub-ppb level concentration of target molecules.

TABLE 2
Summary of Low Concentration Detection of Target Vapor

| Concentration | S₁₁ Average Peak | S₁₁ Std Peak | S₁₁ Peak Amplitude |
|---|---|---|---|
| (ppb) | Amplitude (dB) | Amplitude (dB) | Change (dB) |
| Blank | −41.4 | 0.7 | 0.00 |
| (no vapor) | | | |
| 0.3 | −42.9 | 0.5 | 1.5 |
| 0.6 | −45.5 | 0.5 | 4.1 |
| 1.7 | −44.9 | 0.9 | 3.5 |
| 5.8 | −46.5 | 2.3 | 5.1 |
| 11.5 | −49.5 | 1.9 | 8.1 |

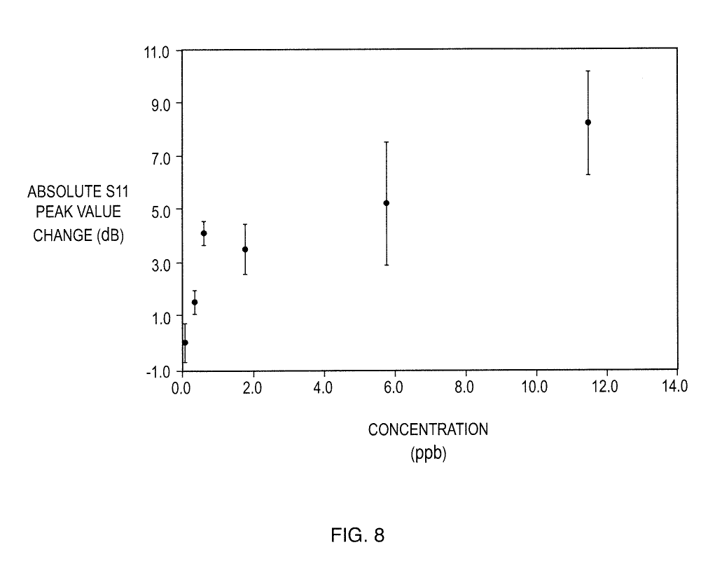

The average S11 amplitude changes are plotted against the target vapor concentration in FIG. 8. The error bars represent the standard deviation of each measurement set. The major contributors to the error in measurements appear to arise from the mechanical disturbance as well as vapor concentration. Efforts to reduce vibrations and mechanical interference were made by using steady connectors to support the cable inside the gas chamber as well as rubber mat. The concentration of gas is controlled by diffusion. The gas vapor was injected to the chamber ˜10 inches away from the sample and allowed to diffuse for 15 seconds. Five measurements were then collected over a period of time ˜100 seconds. It is expected that during this time the vapor has homogeneously diffused inside the chamber volume. However, uncontrolled leaking as well as adsorption/desorption on the surfaces may still be ongoing processes during the measurement collection. The measurements were performed starting from the lowest concentration and the chamber is purged with air in between measurement sets to ensure the recovery of the sensor and removal of the target vapor. As observed in FIG. 8, the absolute S₁₁ peak amplitude increases with increasing concentration of target vapor. At 11.5 ppm, the peak amplitude changed 8.1 dB. The change observed previously with 5 ppm concentration was 10 dB. Comparison of these two results indicates that the sensor film saturates due to the filling up of all the imprinted sites. If required, the dynamic detection range of the sensor could be increased by optimizing parameters such as sample size and density of template molecules on MIP. The density of templates can be increased by adjustment of deposition time and optimizing template solution concentration. The sample size is currently 1 cm*1 cm, which is dependent on the size of the graphene sheet. ACS Materials also provides single sheet graphene with size of 5 cm*5 cm. The sensitivity is expected to be improved with sensor integrated onto a designed antenna.

To demonstrate the specificity of the sensor against possible interfering gas molecules, the methyl salicylate imprinted sensor was tested against water and acetone at high concentration (ppm). The acetone levels observed in atmosphere are 357-2310 parts-per-trillion (ppt),⁶ factor of 1*10⁶ times less than tested here. The observed changes in S₁₁ peak amplitude are below the noise level analyzed from blank (no vapor) measurement which was 0.7 dB. The measurements are summarized in Table 3. The results demonstrate that the sensor is impervious to potential interfering molecules.

TABLE 3
Summary of Test Results on Interfering Gas Molecules

| | Concentration | S₁₁ Peak Amplitude |
|---|---|---|
| Interfering Vapor | (ppm) | Change (dB) |
| Acetone | 300.0 | <0.7 (noise) |
| Acetone | 60.0 | <0.7 (noise) |
| Water | 35 | <0.7 (noise) |

Results on Simulation and Design of Antenna

Return Loss (S₁₁): Return loss is the loss of power in the signal reflected by a discontinuity in a transmission line. This discontinuity can be a mismatch with the terminating load or with a device inserted in the line. It is usually expressed as a ratio in decibels (dB) as defined in Equation (3) and (4)⁷.

$$\begin{matrix}{S}_{11} ( dB ) = 20 {log}_{10} \vert G \vert & ( 3 ) \\ Γ = \frac{{Z}_{L ( f )} - {Z}_{s ( f )}}{{Z}_{L ( f )} + {Z}_{s ( f )}} & ( 4 )\end{matrix}$$

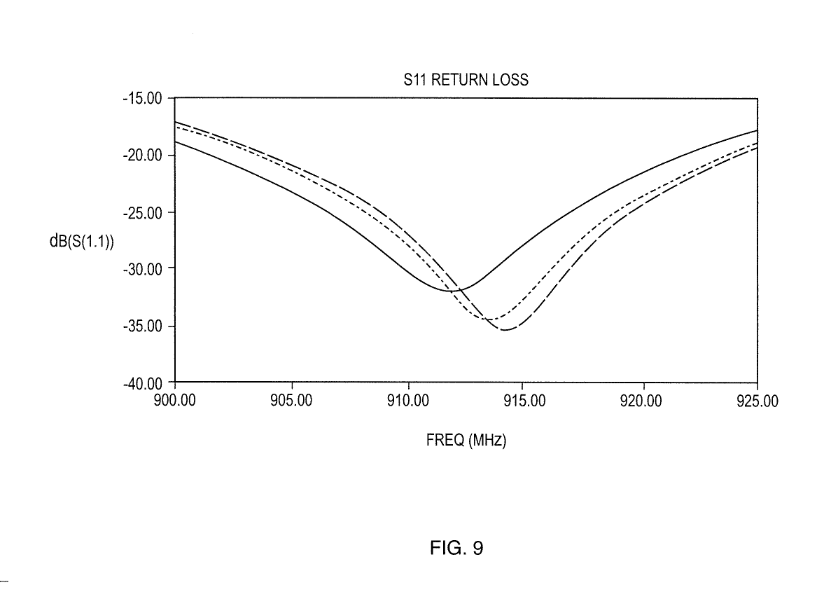

ZS is the impedance toward the source (usually 50Ω) and ZL is the impedance toward the load. Γ is the reflection coefficient which should be matched to 0 (S₁₁ should be as low as possible). Therefore for the antenna design, there is a need to match the load impedance equal to 50Ω. In the simulation, by changing the estimate conductivity (effect the impedance) of the graphene, the S₁₁ change behavior in FIG. 9 can be seen to be similar with the measurements. The simulated frequency shift and amplitude changes are summarized in Table 4.

TABLE 4
Summary of S₁₁ Frequency and Amplitude Shifting
with Changing Conductivity of the Graphene Sheet

| | Peak | Peak | Peak | |
|---|---|---|---|---|
| Conductivity | Frequency | Frequency | Amplitude | Peak Amplitude |
| Change | (MHz) | Shift (MHz) | (dB) | Change (dB) |
| 0% | 911.7 | 0 | −31.94 | 0 |
| −25% | 913.5 | +1.8 | −34.52 | −2.58 |
| −50% | 914.1 | +2.4 | −35.46 | −3.52 |

Results on “In-Situ” Monitoring of Chemical Agent

Integration of MIP-Graphene sensor onto fabricated antenna: Single sheet graphene, purchased from ACS Materials (Medford, Mass.), was directly transferred onto the fabricated antenna. The MIP deposition using malathion as template was then performed under conditions the same as those described previously. An antenna integrated with Graphene-MIP sensor equipped with soldered coaxial ports is shown in FIGS. 5 and 6 and is connected with a network analyzer for testing as shown in FIG. 4b.

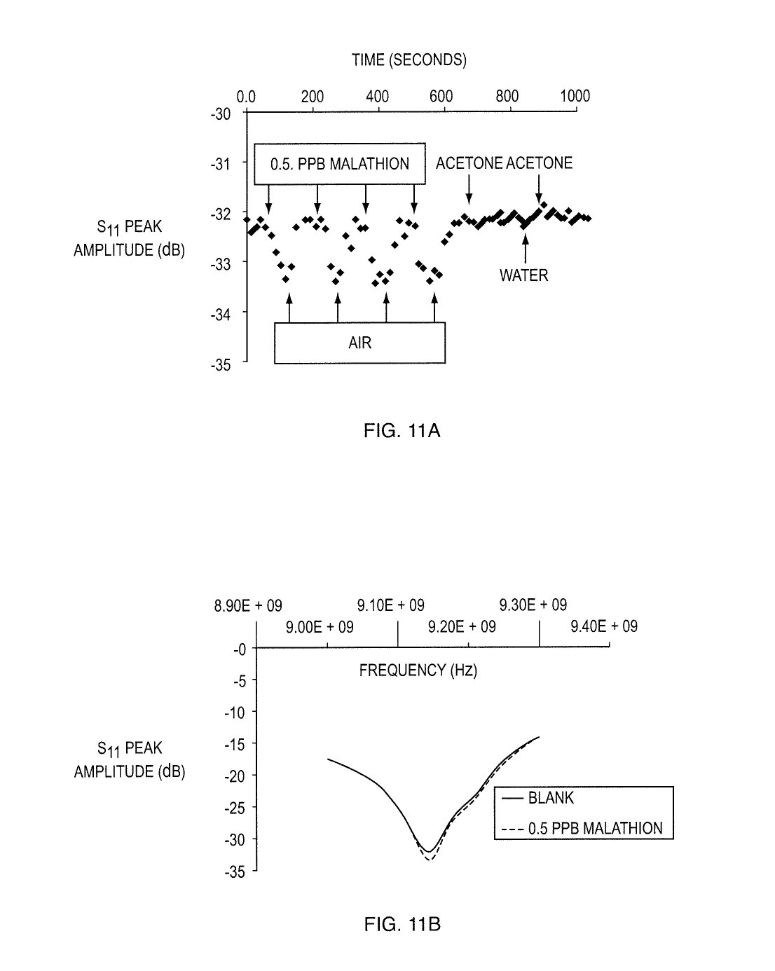

In-situ monitoring of chemical: The resultant antenna with MIP-graphene sensor was characterized. A resonant peak for the sensor was identified at frequency ˜910 MHz, which is within the range of simulation work predicted in Section 4.4. The amplitude in decibel (dB) units was followed before and after addition of gases. FIG. 11b presents the comparison of S₁₁ peak of antenna with graphene-MIP sensor before and after adding 0.5 ppb of malathion vapor. Upon exposure to the target vapor, the power reflection decreased from −32 dB to −33.5 dB. The amplitude change (dB) can be directly related to impedance change of sensor upon rebinding of target molecules. To demonstrate the reversibility of the sensor, the measurements with the network analyzer were performed every 15 seconds during testing. FIG. 11a illustrate a plot of changes in S₁₁ power return loss peak upon addition of analyte-malathion (0.5 ppb), air, and interfering molecules—acetone (300 ppm) and water (35 ppm) as a function of time. As given in FIG. 11a, the blank with no target vapor resulted in peak amplitude of −32 dB while addition of 0.5 ppb malathion resulted in ˜1.5 dB decrease in amplitude to −33.5 dB. After pumping air into the testing chamber, the sensor quickly recovered (within two minutes) in airflow to its original blank amplitude. The specificity of the sensor was also tested against water and acetone at relatively high concentration (ppm). As shown in FIG. 11a, the observed changes in S11 peak amplitude are clearly within the noise fluctuation.

Antenna Gain: Antenna Gain describes how much power is transmitted in the direction of peak radiation to that of an isotropic source. Antenna gain is more commonly quoted in a real antenna's specification sheet because it takes into account the actual losses that occur. From the simulation, our peak gain is 1.95 dB, which is good for an omnidirectional dipole antenna. Dipole antenna gain usually is 0 dB (gain=1) and directional antenna will have higher gain. An antenna with a gain of 1.95 dB means that the power received far from the antenna will be 1.57 times than what would be received from a lossless isotropic antenna with the same input power.

MIP functionalized graphene sensor fabrication and characterization: Optimized conditions for MIP thin layer deposition on single-sheet graphene are presented hereinabove. The graphene-MIP sensor was characterized and the performance was tested against power return loss S₁₁ peak at resonant frequency. The test results demonstrate sensitive sub-ppb level detection of target molecule vapor. The sensor was also exposed to possible interfering vapors (water and acetone). The results indicated that these molecules did not impact the S₁₁ peak amplitude. The results demonstrate that the graphene-MIP sensor has good specificity towards target molecule.

Design and simulation of RF antenna: Design and simulation work for antenna with optimized frequency shift and amplitude change of S₁₁ with effect of impedance change due to detection are presented hereinabove. The designed antenna operates at 902-928 MHz and can radiate and receive RF signal equally well in 360 degree direction. Simulations demonstrate antenna gain of 1.95 dB. The result indicates that the power received far from the antenna will be 1.57 times higher than received from lossless isotropic antenna with the same input power.

For the purposes of describing and defining the present teachings, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Although these teachings have been described with respect to various embodiments, it should be realized these teachings are also capable of a wide variety of further and other embodiments within the spirit and scope of the appended claims.

1. A sensing element for detecting and recognizing target molecules comprising:
a single layer graphene sheet; and
a layer of molecularly imprinted polymer disposed on the single layer graphene sheet; the molecularly imprinted polymer configured to detect and recognize the target molecules.

2. The sensing element of claim 1 further comprising:
electrodes operatively connected to the single layer graphene sheet.

3. The sensing element of claim 1 further comprising:
an antenna operatively connected to the single layer graphene sheet.

4. The sensing element of claim 3 wherein the antenna is a dipole antenna.

5. The sensing element of claim 2 further comprising:
a sensing circuit configured to detect impedance changes in the sensing element.

6. The sensing element of claim 1 wherein the layer of molecularly imprinted polymer comprises a layer of polymerized polyphenol (PPn).

7. The sensing element of claim 1 wherein the layer of molecularly imprinted polymer comprises a layer of polymerized o-Phenylenediamine (OPDA).

8. A backscattering tag comprising the sensing element of claim 1.

9. A method for fabricating a sensing element for detecting and recognizing target molecules, the method comprising:
depositing and imprinting template molecules on a surface of a graphene sheet;
depositing a layer of a monomer on the surface of graphene sheet;
polymerizing the monomer to form a molecular imprinted polymer; and
removing the template molecules.

10. The method of claim 9 further comprising:
attaching electrodes to both ends of the graphene sheet.

11. The method of claim 9 wherein depositing the layer and polymerizing the monomer occur in a same step.

12. The method of claim 11 wherein depositing the layer and polymerizing the monomer occur by electrochemical polymerization.

13. The method of claim 9 wherein depositing and imprinting the template molecules is performed by electrochemical deposition of the template molecules.

14. The method of claim 9 wherein the graphene sheet is a single layer graphene sheet.

15. The method of claim 14 wherein the single layer graphene sheet is transferred to a substrate on which an antenna structure disposed; and wherein the single layer graphene sheet is electrically operatively connected to an antenna.

16. The method of claim 9 wherein the layer of the monomer comprises a layer of polyphenol (PPn).

17. The method of claim 9 wherein the layer of the monomer comprises a layer of o-Phenylenediamine (OPDA).
